(19) United States
(12) Patent Application Publication
(10) Pub. No.: US 2025/0057677 A1
(43) Pub. Date: Feb. 20, 2025

(54) STENT WITH ANTI-MIGRATION FLANGE SUPPORT WITH DELIVERY SYSTEM

(71) Applicant: BOSTON SCIENTIFIC SCIMED, INC., Maple Grove, MN (US)

(72) Inventors: Fionn Stapleton, Galway (IE); John Thomas Favreau, Charlton, MA (US); Ryan Desmond Lynch, Roscommon (IE); David Collins, Galway (IE); John O'Driscoll, Galway (IE); Darren Gerard Curran, Galway (IE); Aran Murphy, Wexford (IE)

(73) Assignee: BOSTON SCIENTIFIC SCIMED, INC., Maple Grove, MN (US)

(21) Appl. No.: 18/799,470

(22) Filed: Aug. 9, 2024

Related U.S. Application Data
(60) Provisional application No. 63/519,375, filed on Aug. 14, 2023.

(51) Int. Cl. A61F 5/00

(52) U.S. Cl. CPC A61F 5/0079

(57) ABSTRACT

Stents and/or implants including a reinforcement member. An illustrative transluminal implant may include an elongated tubular body extending from first end region to a second end region. The elongated tubular body may comprise a scaffolding forming a plurality of cells, a first flange adjacent to the first end region, a second flange adjacent to the second end region, and a saddle region extending between the first flange and the second flange. The saddle region may have an outer diameter less than an outer diameter of the first flange and the second flange. A reinforcement member may be disposed within a lumen of the elongated tubular body and within the first flange.

### CROSS-REFERENCE TO RELATED APPLICATIONS

This application claims the benefit of U.S. Provisional Patent Application Ser. No. 63/519,375, filed Aug. 14, 2023, which is incorporated herein by reference.

### TECHNICAL FIELD

The present disclosure pertains to medical devices, methods for manufacturing medical devices, and uses thereof. More particularly, the present disclosure pertains to a stent for implantation in a body lumen or for transluminal implantation, and associated methods.

### BACKGROUND

A wide variety of intracorporeal medical devices have been developed for medical use, for example, surgical and/or intravascular use. Obesity affects a growing population and may cause additional diseases such as type 2 diabetes, greatly increasing risk of a patient's health. Surgical procedures such as bariatric surgery, e.g., to restrict a portion of a stomach and/or bypass portions of the intestine, may be the only option for patients categorized as morbidly obese. Additionally, these types of procedures may have significant side effects, such as enteric hormonal changes, and are relatively invasive surgical procedures with associated complications, tissue trauma, and/or infections, which in some instances may put the patient at risk. In an alternative procedure, a stent may be used to create an opening between the stomach and the jejunum to allow for the flow of food particulate and liquid from the stomach to the lower GI tract by bypassing the pylorus and duodenum. The stent may be used in conjunction with a device to block the pylorus. Of the known stents, endoluminal implants, and/or transluminal implants, there is an ongoing need to provide alternative configurations of stents, endoluminal implants, and/or transluminal implants.

### SUMMARY

This disclosure provides design, material, manufacturing method, and use alternatives for medical devices. An example medical device may include a stent.

In a first example, a transluminal implant may comprise an elongated tubular body extending from first end region to a second end region. The elongated tubular body may comprise a scaffolding forming a plurality of cells, a first flange adjacent to the first end region, a second flange adjacent to the second end region, and a saddle region extending between the first flange and the second flange, the saddle region having an outer diameter less than an outer diameter of the first flange and the second flange. A reinforcement member may be disposed within a lumen of the elongated tubular body and within the first flange, the reinforcement member comprising at least one curved wire.

Alternatively or additionally to any of the examples above, in another example, the reinforcement member may be configured to exert a radially outward force on the elongated tubular body.

Alternatively or additionally to any of the examples above, in another example, an outer diameter of the reinforcement member may be similar to an outer diameter of the first flange.

Alternatively or additionally to any of the examples above, in another example, an outer diameter of the reinforcement member may be greater than an outer diameter of the first flange.

Alternatively or additionally to any of the examples above, in another example, the at least one curved wire may include at least one winding extending about an inner surface of the first flange.

Alternatively or additionally to any of the examples above, in another example, the at least one curved wire may include at least two windings extending about an inner surface of the first flange.

Alternatively or additionally to any of the examples above, in another example, the at least one curved wire may include less than one winding.

Alternatively or additionally to any of the examples above, in another example, the reinforcement member may comprise two or more curved wires.

Alternatively or additionally to any of the examples above, in another example, the at least one curved wire may have a diameter of about 0.013 inches (0.330 millimeters).

Alternatively or additionally to any of the examples above, in another example, a proximal end of the at least one curved wire may be secured to a distal end of the at least one curved wire.

Alternatively or additionally to any of the examples above, in another example, the at least one curved wire may comprise a shape memory material.

Alternatively or additionally to any of the examples above, in another example, the transluminal implant may further comprise a second reinforcement member disposed within the lumen of the elongated tubular body and within the second flange.

Alternatively or additionally to any of the examples above, in another example, the elongated tubular body may comprise a stent.

Alternatively or additionally to any of the examples above, in another example, the elongated tubular body may comprise a pyloric closure device.

Alternatively or additionally to any of the examples above, in another example, the reinforcement member may further comprise a hardening material.

In another example, a transluminal implant may comprise an elongated tubular body extending from first end region to a second end region and comprising a scaffolding forming a plurality of cells, a first flange adjacent to the first end region, a second flange adjacent to the second end region, and a saddle region extending between the first flange and the second flange, the saddle region having an outer diameter less than an outer diameter of the first flange and the second flange and a self-expanding reinforcement member disposed against an inner surface of the elongated tubular body within the first flange. The self-expanding reinforcement member may be configured to exert a radially outward force on the elongated tubular body.

In another example, a delivery system for delivering a reinforcement member to an implant may comprise an endoscope including a working channel, a delivery shaft movably disposed within the working channel, a reinforcement member disposed within a lumen of the delivery shaft and a pusher element movably disposed within the lumen of the delivery shaft, a distal end of the pusher element configured to engage a proximal end of the reinforcement member.

Alternatively or additionally to any of the examples above, in another example, the delivery shaft may have a stiffness greater than a stiffness of the reinforcement member.

Alternatively or additionally to any of the examples above, in another example, the reinforcement member may be configured to move from an elongated delivery configuration to a curved deployed configuration as the reinforcement member is distally advanced from the delivery shaft.

Alternatively or additionally to any of the examples above, in another example, the delivery shaft may include a curved distal end region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side view of an illustrative stent;

FIG. 2 is a cross-sectional view of the illustrative stent of FIG. 1;

FIG. 3 is a schematic view of the illustrative stent of FIG. 1 implanted in a body;

FIG. 4 is a side view of an illustrative pyloric closure device;

FIGS. 5A and 5B are a perspective view of an illustrative reinforcement member;

FIG. 6 is a perspective view of an illustrative stent including a reinforcement member disposed within a flange thereof; and

FIGS. 7A and 7B illustrate schematic partial cross-sectional views of an illustrative system and method for delivering the reinforcement member.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

In some cases, it may be desirable to use lumen apposing metal stents (LAMS) for novel metabolic endoscopy and natural orifice transluminal endoscopic surgery (NOTES) to form an anastomosis (gastrojejunostomy) between the stomach and jejunum. This facilitates the flow of food particulate and liquid from the stomach to the lower GI tract by bypassing the pylorus and duodenum. In conjunction with a device to block the pylorus, a stent, such as, but not limited to, an AXIOS™ lumen apposing metal stent made and distributed by Boston Scientific, Corporation, has been used in preclinical studies. These devices (e.g., the device blocking the pylorus and the stent) may be intended to be used to treat obesity by bypassing the first 1.5 meters of the small intestine, where most food fats and nutrients are digested. In this manner, stomach contents (e.g., food and other nutrients) may not be absorbed as it travels from the stomach through the distal small intestine, promoting patient weight loss and possibly controlling type-2 diabetes. This procedure may be used as a less invasive treatment for obesity and type-2 diabetes, which may be typically treated with Roux-en-Y surgical bypass.

The current stent design may include a fully silicone coated nitinol metal stent with a proximal and distal flange either side of the saddle region which promotes the formation of the anastomosis. The stent may be fully coated to prevent leakage of the food particles and/or liquid during anastomosis formation. On some occasions when the anastomosis is formed migration of the stent has been observed on the animal model. Based on initial preclinical studies, a 33% migration rate has been observed on the animal model using the current stent design. Indications for in-dwell duration for a LAMS may be moving from maximum of 60 days to upwards of 6-12 months, or more. What may be desirable is a LAMS, or other transluminal implant, with more robust antimigration features. Disclosed herein are devices and systems which provide additional pull-out force resistance. While the present disclosure is discussed with respect to transluminal implants for forming an anastomosis, it should be understood that the devices described herein may be endoluminal implants as well. Further, the implant location is not limited to a particular anatomical location.

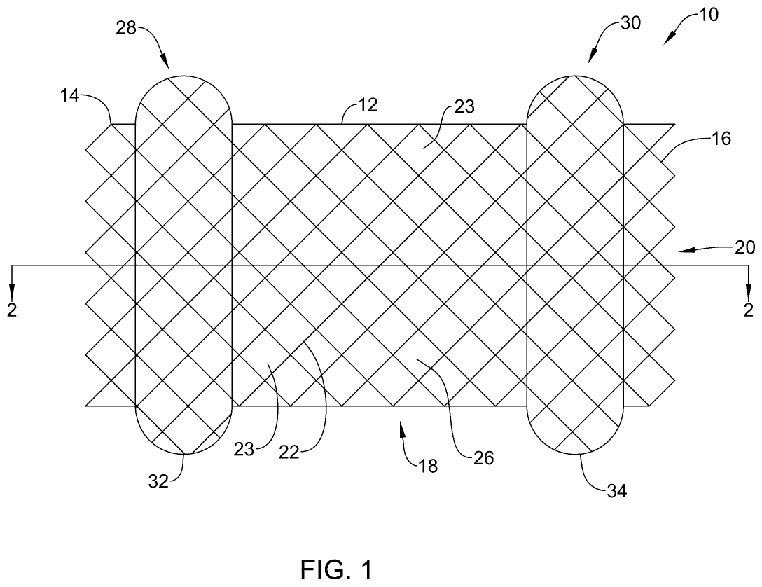

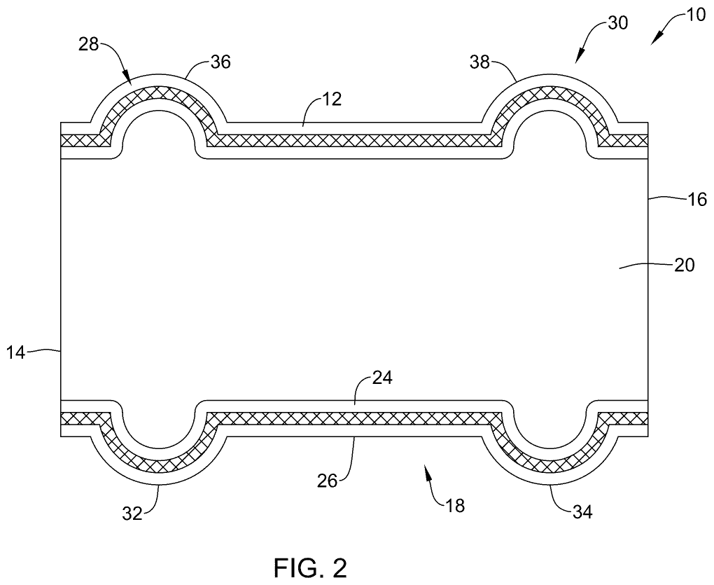

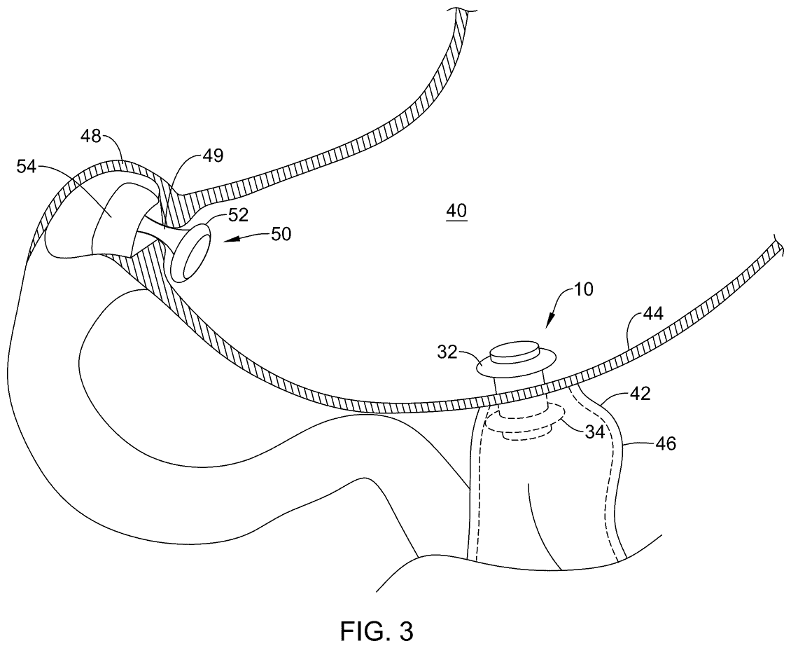

FIG. 1 illustrates a side view of an illustrative implant 10, such as, but not limited to, a stent, in an expanded state. FIG. 2 illustrates a cross-sectional view of the illustrative stent 10, taken at line 2-2 of FIG. 1. FIG. 3 illustrates a schematic partial cross-sectional view of the illustrative stent 10 implanted in the body of a patient to form an anastomosis. In some instances, the stent 10 may be formed from an elongated tubular member 12. While the stent 10 is described as generally tubular, it is contemplated that the stent 10 may take any cross-sectional shape desired. The stent 10 may have a first, or proximal, end 14, a second, or distal, end 16, and an intermediate region 18 disposed between the first end 14 and the second end 16. In some cases, the intermediate region 18 may be referred to as a saddle region. The stent 10 may include a lumen 20 extending from a first opening adjacent the first end 14 to a second opening adjacent to the second end 16 to allow for the passage of food, fluids, etc. therethrough.

The stent 10 may be radially expandable from a first radially collapsed configuration (not explicitly shown) to a second radially expanded configuration, as shown in FIGS. 1-3. The stent 10 may be structured to extend across two non-adherent structures/tissues and to apply a radially outward pressure to create an opening or passage between the two non-adherent structures/tissues, thereby forming an anastomosis between the two separate anatomical structures.

The tubular member 12 of the stent 10 may have a scaffold structure, fabricated from one or more, or a plurality of interwoven filaments or struts 22. The scaffold structure may extend from the first end 14 to the second end 16 of the stent 10. For example, the scaffold structure, and thus the filament(s) thereof, may extend continuously from the first end 14 to the second end 16 of the stent 10. In some embodiments, the stent 10 may be formed with one filament interwoven with itself (e.g., knitted) to form the scaffold structure. In other embodiments, the stent 10 may be formed with several interwoven filaments (e.g., braided) to form the scaffold structure. Thus, in such instances one or more of the filament(s) forming the scaffold structure may extend continuously from the first end 14 to the second end 16 of the stent 10. In still another embodiment, the stent 10 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 14 to the second end 16 of the stent 10.

In some instances, an inner and/or outer surface of the scaffold structure of the stent 10 may be entirely, substantially or partially, covered with a polymeric covering or layer 24, 26 (see, for example, FIG. 2). For example, a covering or coating may extend across the open cells of the scaffold structure to prevent tissue ingrowth into the lumen of the stent 10. However, in some embodiments one or both of the polymeric coverings 24, 26 may be omitted. For example, in some embodiments the stent 10 may include only the outer polymeric covering 26 on an outer surface of the scaffold structure. In other embodiments the stent 10 may include only the inner polymeric covering 24 on an inner surface of the scaffold structure. In some instances, the inner layer 24 and the outer layer 26 may be formed as a single unitary structure. In other embodiments, the inner layer 24 and the outer layer 26 may be formed as separate layers. The inner and outer layers 24, 26 may be formed from the same material or different materials, as desired. The inner layer 24 and/or outer layer 26 may span or be disposed within openings or interstices defined between adjacent stent filaments or struts 22 of the scaffold structure. It can be appreciated that as inner layer 24 and outer layer 26 extend outwardly and inwardly, respectively, they may touch and/or form an interface region within the spaces (e.g., openings, cells, interstices) 23 in the wall of the scaffold structure of the stent 10. For example, the inner and outer layers 24, 26 may extend into the openings 23 defined between adjacent stent struts 22 and form an interface region. Further, the inner and outer layers 24, 26 may additionally extend between adjacent filaments or struts 22, thereby filling any space between adjacent filaments or strut members 22, and thus prevent tissue ingrowth into the lumen of the stent 10.

It is contemplated that the scaffold structure, e.g., the filaments and/or struts, of the stent 10 can be made from a number of different materials such as, but not limited to, metals, metal alloys, shape memory alloys, and/or polymers, as desired, enabling the stent 10 to be expanded into shape when accurately positioned within the body. For example, while lumen apposing metal stents have been referenced herein, it is not required that the stent 10 be formed from a metal or metal alloy. In some instances, the material may be selected to enable the stent 10 to be removed with relative ease as well. For example, the stent 10 can be formed from alloys such as, but not limited to, nitinol and Elgiloy®. Depending on the material selected for construction, the stent 10 may be self-expanding or require an external force to radially expand the stent 10. In some embodiments, filaments may be used to make the stent 10, which may be composite filaments, for example, having an outer shell made of nitinol and having a platinum core. It is further contemplated the filaments of the stent 10 may be formed from polymers including, but not limited to, polyethylene terephthalate (PET).

In some instances, in the radially expanded configuration, the stent 10 may include a first end region 28 proximate to the first end 14 and a second end region 30 proximate to the second end 16. In some embodiments, the first end region 28 and the second end region 30 may include shoulders or enlarged regions, such as flanges 32, 34 positioned adjacent to the first end 14 and the second end 16 of the stent 10. The flanges 32, 34 may be configured to engage an interior portion of the walls of the body cavity or body lumen. For example, the first flange 32 may be positioned against an interior of a first body lumen and the second flange 34 may be positioned against an interior of a second body lumen different from the first body lumen. Thus, the stent 10 may be positioned to traverse between two separate anatomical structures. For example, referring to FIG. 3, the stent 10 is positioned such that it extends between the stomach 40 and the jejunum 42. The first flange 32 may be positioned in the stomach 40 and the second flange 34 may be positioned in the jejunum 42. The intermediate region or saddle 18 of the stent 10 extending between the first flange 32 and the second flange 34 may extend through the wall 44 of the stomach 40 and the wall 46 of the jejunum 42, interconnecting the two anatomical structures. In some cases, the first flange 32 may contact an interior of the wall 44 of the stomach 40 and/or the second flange 34 may contact an interior of the wall 46 of the jejunum 42. The first and/or second flanges 32, 34 may anchor the stent 10 against the stomach wall and the jejunum all 44 so that stent 10 is inhibited or prevented from migrating. However, this is not required.

The flanges 32, 34 may extend circumferentially around the stent 10, and define an annular pocket extending circumferentially around an interior of the stent 10. The annular pocket may have an enlarged inner diameter relative to portions of the flange 32, 34 on either side thereof. In some embodiments, the flanges 32, 34 may have a larger diameter than the intermediate region or saddle 18 of the stent 10 located between the end regions 28, 30 to prevent or help prevent the stent 10 from migrating once placed within a body cavity, body lumen, or across body cavities or lumens. It is contemplated that the transition from the cross-sectional area of the intermediate region or saddle 18 to the retention features or flanges 32, 34 may be gradual, sloped, or occur in an abrupt step-wise manner, as desired. In some cases, the flanges 32, 34 may have a curved semi-hemispherical shape that gradually increases in cross-sectional dimensions and then gradually decreases in cross-sectional dimension in a direction such that the first and/or second ends 14, 16 have a similar cross-sectional dimension to the intermediate region or saddle 18. However, this is not required. Other shapes and/or configurations may be used, as desired. For example, in some cases, a distal side 36 of the proximal flange 32 and a proximal side 38 of the distal flange 34 may have a generally concave shape.

In some embodiments, the first flange 32 may have a first outer diameter and the second flange 34 may have a second outer diameter. The outer diameter of the first flange 32 and/or the second flange 34 may be greater than the outer diameter of the intermediate region or saddle 18. The inner diameter of the first flange 32 and/or the second flange 34 may be greater than the inner diameter of the intermediate region or saddle 18. In some instances, the first flange 32 may have an inner diameter greater than the inner diameter at the first end 14 of the stent 10 and/or the second flange 34 may have an inner diameter greater that the inner diameter at the second end 16 of the stent 10. Thus, the first flange 32 may have a greater inner diameter than portions of the stent 10 extending in opposite directions from the first flange 32 and/or the second flange 34 may have a greater inner diameter than portions of the stent 10 extending in opposite directions from the second flange 34. In some instances, the first and second outer diameters may be approximately the same, while in other instances, the first and second outer diameters may be different. In some embodiments, the stent 10 may include only one flange 32, 34, or the stent 10 may not include a flange, if desired. For example, the first end region 28 may include a flange 32 while the second end region 30 may have an outer diameter similar to that of the intermediate region or saddle 18. It is further contemplated that the second end region 30 may include a flange 34 while the first end region 28 may have an outer diameter similar to that of an outer diameter of the intermediate region or saddle 18. In some embodiments, the stent 10 may have a uniform outer diameter from the first end 14 to the second end 16. In some embodiments, the outer diameter of the intermediate region or saddle 18 may be in the range of 15 to 25 millimeters. The outer diameter of the flanges 32, 34 may be in the range of 20 to 30 millimeters. It is contemplated that the outer diameter of the stent 10 may be varied to suit the desired application.

Referring to FIG. 3, the stent 10 may be placed between the stomach 40 and the jejunum 42 using a delivery system. Some illustrative delivery systems and methods of delivery are described in commonly assigned U.S. Patent Publication Number 2019/0298401, filed Mar. 22, 2019, entitled “Systems and Methods for Performing Endoscopic Procedures,” which is herein incorporated by reference in its entirety. In some cases, the stent 10 may be used in conjunction with another endoluminal or transluminal implant 50. For example, the implant 50 may be a pyloric closure device. The pyloric closure device 50 may extend across the pylorus from the stomach 40 to the duodenum 48.

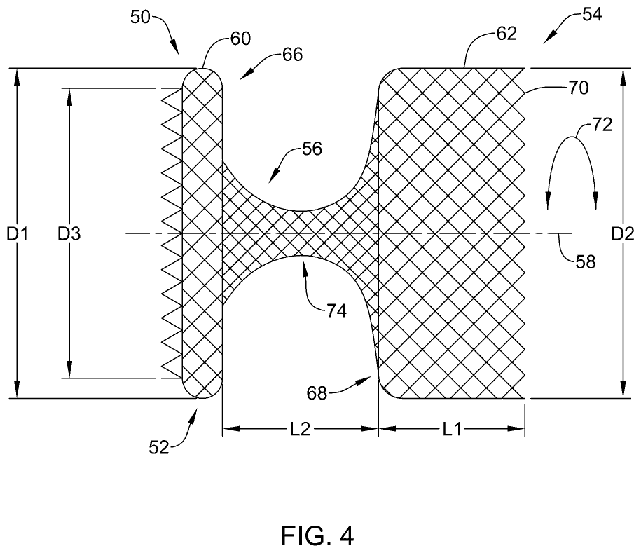

FIG. 4 is a side view of an illustrative pyloric closure device 50. Alternative pyloric closure devices are described in commonly assigned U.S. Patent Publication Number 2021/0244523, filed Apr. 28, 2021, entitled “Devices, Systems, and Methods for Pyloric Occlusion,” U.S. Patent Publication Number 2022/0346996, filed Apr. 27, 2022, entitled “Devices, Systems, and Methods for Duodenal Exclusion and Stomach Capacity Reduction,” and U.S. Patent Publication Number 2023/0165589, filed Nov. 22, 2022, entitled “Devices, Systems, and Methods for Pyloric Occlusion,” which are herein incorporated by reference in their entirety. In some examples, the pyloric closure device 50 may be formed as a closed stent. The pyloric occlusion device 50 (e.g., a pyloric closure stent) may include a first flange 52 and a second flange 54 connected by a saddle 56, having a lumen extending along longitudinal axis 58. It may be understood that the flanges 52, 54 may be any configuration so that a diameter of the flanges 52, 54 is greater than a diameter of the saddle 56. For example, the flanges 52, 54 may include any number of configurations, orientations, shapes, tapers, step ups, inflection points, etc. The pyloric occlusion device 50 may further include a closure element, as will be described in more detail herein.

The first flange 52, which may extend circumferentially around the stent 10, may be a bulb, e.g., having a rounded portion 60, expanding radially with respect to the longitudinal axis 58. The bulb and/or extension may have a curvature to define a circumference. The bulb may be formed as circular, spherical, semi-spherical, and/or elliptical, although other shapes are also envisioned. In some embodiments, the first flange 52 may include a rounded portion 60, which may minimize tissue damage (e.g., perforation). In some embodiments, an edge 64 may extend from the first flange 52 in a direction opposite from a distal surface 66 for manufacturing and/or for loading into the constrained position in a delivery device. The second flange 54 may be formed as a duodenal extension 62. The second flange 54 may be generally tubular (e.g., as an open stent) and may have a length L1 extending from a proximal surface 68 in a longitudinally outward direction along the axis 58. The duodenal extension 62 may extend a length L1 to an edge 70. The duodenal extension 62 may provide a holding force in the duodenum.

The first flange 52 may have a first diameter D1 and the second flange 54 may have a second diameter D2. In some embodiments, the first and second diameters D1 and D2 may be equal, although in other embodiments, the first and second diameters D1 and D2 may be different. It is understood that the first and second diameters D1 and D2 may be any size that exceeds the size of the opening of the pylorus, e.g., so the first and/or second flanges 52, 54 may contact the respective gastric wall and/or duodenum wall. The edge 64 of the first flange 52 may form a diameter D3, which may be less than first diameter D1.

The saddle 56 may connect the first and second flanges 52, 54. In some embodiments, the saddle 56 may be a hollow tube, which is then formed in a twisted shape to close the saddle 56. For example, a closure element may be configured to occlude a flow of material (e.g., stomach contents including food, liquid, and/or nutrients) through the saddle 56. The saddle 56 may be twisted, or rotated, about the longitudinal axis 58 as indicated by arrow 72 to create a kink, or closure element 74. In some embodiments, one of the first or second flanges 52, 54 may be rotated relative to the other of the first or second flange 52, 54, to create the closure element 74 in the saddle 56. In some embodiments, the first and second flanges 52, 54 may both rotate relative to each other in opposite directions to create the closure element 74 in the saddle 56. The rotation may be any amount to fully occlude the saddle 56 (e.g., approximately 180° to approximately 720°). The pyloric occlusion device 50 may be formed of a braided nitinol material, which, when twisted, may result in a high density of braided material or wires in the channel of the saddle 56 to form the occlusion. For example, the braided material may be concentrated at the closure element 74, and/or may have a higher density of material at the closure element 74.

The saddle 56 may be any length, indicated as L2, required to traverse the pyloric sphincter. In some embodiments, the saddle 56 may be in the range of about 5 millimeters (mm) to about 25 mm, although it is envisioned that the saddle 56 may be any length, including less than 5 mm and/or greater than 25 mm to perform the desired procedure on a patient.

The nitinol braiding may be heat-set, so that the closure element 74 is pre-formed in the pyloric occlusion device 50. For example, as the pyloric occlusion device 50 is deployed, the saddle 56 may expand including the closure element 74, so that a portion of the pyloric occlusion device 50 is rotated as it is deployed and self-expands to the pre-set shape. It is also understood that in some embodiments, a pyloric occlusion device 50 may not be pre-set, so that the medical professional may partially deploy the pyloric occlusion device 50. For example, a portion of the pyloric occlusion device may be expanded (e.g., the second flange 54 and at least a portion of the saddle 56) while the remaining portion of the pyloric occlusion device remains in the delivery system. The medical professional may then rotate the delivery system to create a closure element 74 in the saddle 56. The remaining portion of the pyloric occlusion device 50 may then be delivered to expand the remaining portion of the saddle 56 and the first flange 52.

When deployed (see FIG. 3), an inner or proximal surface 68 of the second flange 54 may contact a surface of the duodenum wall 49, for example, so that the second flange is deployed against the duodenum wall. Additionally, an inner or distal surface 66 of the first flange 52 may contact a surface of the stomach wall 44, for example, so that the first flange is deployed against the stomach wall 44. The saddle 56 including the closure element 74 may extend between the duodenum wall 49 and the stomach wall 44 through the pylorus to block, or occlude, the pylorus.

The first and/or second flanges 52, 54 may anchor the device against the duodenum wall 49 and the stomach wall 44 when the respective surfaces 66, 68 contact the walls 44, 49 so that pyloric occlusion device 50 is inhibited or prevented from migrating. In some embodiments, the first and/or second flanges 52, 54 may be attached to the walls 44, 49 by mechanical fasteners such as clips, sutures, and the like.

In some embodiments, pyloric occlusion device 50 may be fully coated, to minimize and/or prevent tissue ingrowth, and in other embodiments, a portion of the pyloric occlusion device 50 may be uncoated and other portions of the device 50 may be coated. An uncoated device 50 may be advantageous to later remove the pyloric occlusion device with, for example, an argon plasma coagulation (APC) device, from the patient without having to disengage from duodenum and/or gastric tissue. In some embodiments, the saddle 56 may be uncoated to promote tissue ingrowth, which may be advantageous to anchor (e.g., minimizing and/or preventing migration) the device 50 in tissue of the pylorus.

In some embodiments, a coating may be applied to the first and/or second flanges 52, 54, while the saddle 56 remains uncoated. A coating (not explicitly shown) may be deposited on at least a portion of the first and/or second flanges 52, 54, and may penetrate through the braiding. In other embodiments, one or more coating may extend along an entirety of a length of pyloric closure device 50. The coating(s) may not affect the total length of the device 50 which may be in the range of about 10 mm to about 50 mm.

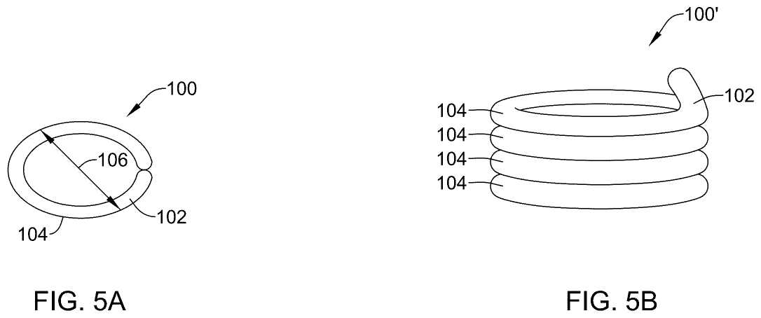

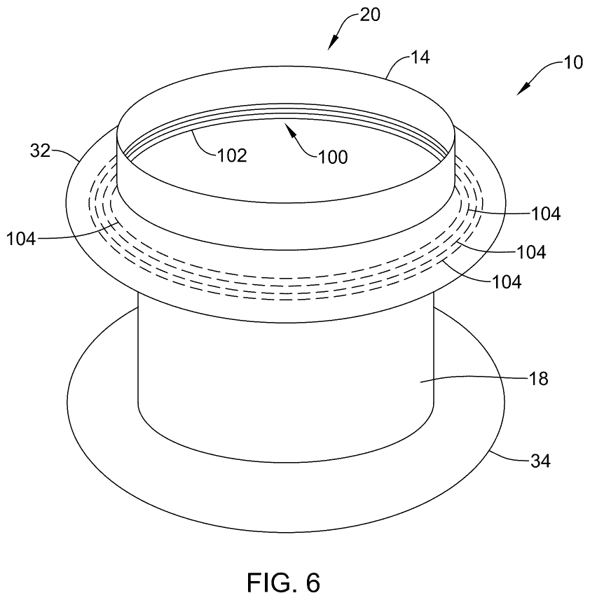

FIGS. 5A and 5B are a perspective view of an illustrative reinforcement member 100, 100′ that may be used with the stent 10 and/or pyloric closure device 50 described herein. While the reinforcement member 100 is described with respect to the stent 10 and/or pyloric closure device 50, it should be understood that the reinforcement member 100 may be used in other stents, endoluminal implants, transluminal implants, etc., to reduce the risk of migration. Generally, the reinforcement member 100 may be configured to be positioned within a lumen of the expanded stent 10 and/or pyloric closure device 50 to provide additional pull-out force resistance to prevent migration of the stent 10 and/or pyloric closure device 50. For example, the reinforcement member 100 may be positioned within the annular pocket defined by the proximal flange 32 of the stent 10 to prevent migration of the stent 10 from the stomach 40 to the jejunum 42. In another example, the reinforcement member 100 may be positioned within the first, or proximal, flange 52 of the pyloric closure device 50 to prevent migration of the pyloric closure device 50 from the stomach 40 to the duodenum 48.

The reinforcement member 100 may include a wire 102 that has been wound or curved into a coil. The coil may include any number of whole or partial windings 104 desired. As used herein, a winding may be a complete 360° loop of the wire 102. FIG. 5A illustrates a reinforcement member 100 that has about one complete winding 104. FIG. 5B illustrates a reinforcement member 100′ that includes at least four complete windings 104. It is contemplated that the reinforcement member 100 may include less than one complete winding 104 (e.g., extending less than 360°), more than one complete winding 104, more than two windings 104, more than four windings 104, etc. In some examples, more than one wire 102 may be used to form the reinforcement member 100. For example, a reinforcement member 100′ having four windings 104 may be formed from four individual reinforcement members 100 having about one complete winding each. This is just one example. It is contemplated that any number of wires 102 having any number of complete (e.g., 360° or more) or partial arc lengths (e.g., less than 360°) may be used to form the reinforcement member 100. Further, while FIG. 5B illustrates the windings 104 of the reinforcement member 100′ as being laterally adjacent to one another (e.g., with an edge of a winding contacting an edge of an adjacent winding), this is not required. Additional windings may be positioned radially inward, radially outward, and/or longitudinally offset from one another. It is further contemplated that the windings 104 may not necessarily contact one another.

The windings 104 may have an outer diameter 106 that is approximately the same as the outer diameter of the flange (or another portion of the implant) in which it is placed. In other examples, the windings 104 may have an outer diameter 106 that is greater than the outer diameter of the flange (or another portion of the implant) in which it is placed with the stent 10 or pyloric device 50 in its expanded state. In yet other examples, the windings 104 may have an outer diameter 106 that is less than the outer diameter of the flange (or another portion of the implant) in which it is placed.

When positioned within a flange 32, 34 of the stent 10, the windings 104 may have an outer diameter of about 28 mm to about 32 mm, for example. Manufacturing the reinforcement member 100 to have a same or similar outer diameter as its intended placement location may allow the reinforcement member 100 to provide an additional radial outward force to the expanded stent 10 or pyloric closure device 50. This additional radial force may make it more difficult for the stent 10 or pyloric closure device 50 to migrate during peristalsis or turbulence caused by the digestion of a food bolus. Said differently, the reinforcement member 100 may reinforce the stent 10 and/or pyloric closure device 50 to help prevent compression of the stent 10 and/or pyloric closure device 50 and subsequent migration thereof. This in turn may allow for a longer in-dwell duration for the stent 10 and/or pyloric closure device 50 during a metabolic procedure. It is further contemplated that positioning and/or sizing the reinforcement member 100 to rest against an inner surface of the flange 32, 34 may position the reinforcement member 100 such that it does not extend radially into the inner diameter of the saddle portion 18. This may prevent the reinforcement member 100 from impacting or interfering with the flow of food, fluids, and/or chyme from the stomach 40 to the jejunum 42.

The wire 102 may have a generally circular cross-sectional shape. However, the wire 102 may have other cross-sectional shapes, such as, but not limited to, square, rectangular, polygonal, triangular, oblong, hemispherical etc. The wire 102 may have a diameter of about 0.013 inches (0.330 mm). However, the diameter may be less than 0.013 inches (0.330 mm) or greater than 0.013 inches (0.330 mm), as desired.

It is contemplated that the reinforcement member 100 may be formed from a number of different materials such as, but not limited to, metals, metal alloys, shape memory alloys and/or polymers, as desired, enabling the reinforcement member 100 to be biased into a linear configuration for delivery and a coiled shape when the reinforcement member 100 is in use. Depending on the material selected for construction, the reinforcement member 100 may be self-expanding (i.e., configured to automatically expand when unconstrained). As used herein the term “self-expanding” refers to the tendency of the reinforcement member 100 to return to a preprogrammed shape when unrestrained from an external biasing force (for example, but not limited to, a delivery device, etc.). For example, the reinforcement member 100 may be heat set into a coil configuration and elongated into a linear configuration for delivery through a delivery device. As the reinforcement member 100 exits the delivery device, the reinforcement member 100 may resume the coiled configuration. In some embodiments, the wire 102 may be a nitinol wire. However, this is not required.

It is contemplated that the reinforcement member 100 may include features which limit potential damage to the stent 10, pyloric closure device 50, and/or surrounding tissues. For example, the endpoints (e.g., a proximal end and a distal end of the wire 102) of the wire 102 may be rounded or otherwise shaped to be atraumatic which may prevent damage to the stent, pyloric closure device 50, and/or surrounding tissues. In some cases, one or both of the endpoints may be curved to extend radially inward away from the inner surface of the stent 10, pyloric closure device 50, and/or surrounding tissues. In another example, the endpoints of the wire 102 may include a cap formed from a material softer than the wire 102 to protect the stent 10, pyloric closure device 50, and/or surrounding tissues. In a further example, the endpoints of the wire 102 may be secured to one another using a tubular end cap component or through welding, brazing, soldering, etc. In some embodiments, the endpoints of the wire 102 may be secured to one another prior to delivering the reinforcement member 100 to the stent 10 or pyloric closure device 50. For example, the reinforcement member 100 may be a complete loop which is pinched together to deliver portions of the reinforcement member 100 side-by-side within the delivery system.

As described above, the reinforcement member 100 may be configured to be disposed within a flange 32, 34 of the stent 10 or a flange 52, 54 of the pyloric closure device 50. The reinforcement member 100 may be configured to be disposed within a flange 32, 34 of the expanded stent 10 or a flange 52, 54 of the expanded pyloric closure device 50 subsequent to deployment and expansion of the stent 10 or the pyloric closure device 50. Thus, the reinforcement member 100 may be separate from the stent 10 or the pyloric closure device 50, and intraoperatively deployed in the expanded stent 10 or pyloric closure device 50. For example, after the stent 10 or pyloric closure device 50 is delivered, the reinforcement member 100 may be delivered within the lumen of the stent 10 or pyloric closure device 50 to an interior annular pocket of one of the flanges 32, 34, 52, 54. FIG. 6 is a perspective view of the illustrative stent 10 including a reinforcement member 100 disposed within the proximal flange 32. For ease of illustration and to facilitate understanding of the placement of the reinforcement member 100, the struts 22 of the stent 10 have been omitted in FIG. 6. As can be seen in FIG. 6, the reinforcement member 100, including a plurality of windings 104, has been positioned within the lumen 20 of the stent 10 and longitudinally adjacent to the proximal flange 32. For example, the reinforcement member 100 is radially inward of an outer surface of the expanded stent 10 so as to provide a radially outward force thereon. In some embodiments, a reinforcement member 100 may be additionally or alternatively provided within the distal flange 34. It is contemplated that a reinforcement member 100 may be positioned within a flange 52, 54 of the pyloric closure device 50 in a similar manner.

It is contemplated that increasing the stiffness of the reinforcement member 100 may increase the pull-out force required to pull the stent 10 and/or pyloric closure device 50 through the orifice through which it extends. It is contemplated that increasing the number of windings 104 of the reinforcement member 100 may increase the radial strength and thus the pull-out force of the device in which the reinforcement member 100 is disposed. Additionally or alternatively to increasing the stiffness of the reinforcement member 100 by increasing the number of windings 104, in some cases, the stiffness of the reinforcement member 100 may be increased by increasing a diameter, or cross-sectional dimension, of the wire 102. It is further contemplated that the diameter, or cross-sectional dimension, of the wire 102 may be increased or decreased to allow the reinforcement member 100 to fit within the channel of the flange 32, 34.

To simulate device migration and to determine the increase in pull-out force provided by a reinforcement member 100, an expanded stent, similar to stent 10, was positioned through two pieces of silicone having holes extending therethrough to simulate an anastomosis in the body. The stent included a control device which included no reinforcement member. A plurality of reinforcement members having one coil, two coils, four coils, six coils, and eight coils were positioned in the proximal flange of a plurality of stents. A pulling force was exerted on the distal end (e.g., on an opposing side of the silicone from the reinforcement member) of the stent. The force which caused to the stent 10 to pass through the holes of the silicone was recorded as the pull-out force. Table 1 below summarizes the experimental data.

TABLE 1
Pull-Out Force Data

| Type of Reinforcement member | Pull-Out Force (N) |
|---|---|
| Control (no reinforcement member) | 4.105 |
| 1 coil | 7.33 |
| 2 coils | 12.324 |
| 4 coils | 12.308 |
| 6 coils | 17.142 |
| 8 coils | 28.45 |

As can be seen, the control stent had a pull-out force of about 4.105 Newtons (N). Generally, as the number of windings increased, the pull-out force required to dislodge the stent also increased. Adding a reinforcement member with a single coil demonstrated an 81% increase in the pull-out force required to dislodge the stent while a reinforcement member with eight coils demonstrated a 690% increase in the pull-out force required to dislodge the stent. Thus, the number of windings 104 or coils of the reinforcement member 100 may be increased or decreased to increase or decrease the pull-out force.

It is contemplated that a reinforcement member 100 having eight windings 104 formed from a single continuous wire 102 may have a length that is eight times as long as a reinforcement member 100 having a single winding 104. As such, longer delivery devices may be required to deploy reinforcement members 100 having more windings 104. However, as described herein, in some cases, the windings 104 may be formed from a plurality of shorter individual wires 102. Thus, the reinforcement member 100 may include a plurality of shorter wires deployed sequentially through a shorter delivery device compared to a length of a delivery device required for a reinforcement member 100 having a similar number of windings 104 but is formed from a single wire 102.

In addition to or in place of the reinforcement member 100, a hardening material may be disposed within the flange 32, 34 to increase the strength of the flange 32, 34. It is contemplated that after delivery of the stent 10, a hardening material such as silicone, hydrogels, cyanoacrylates, or other biocompatible adhesives may be dispensed into one or both of the flanges 32, 34.

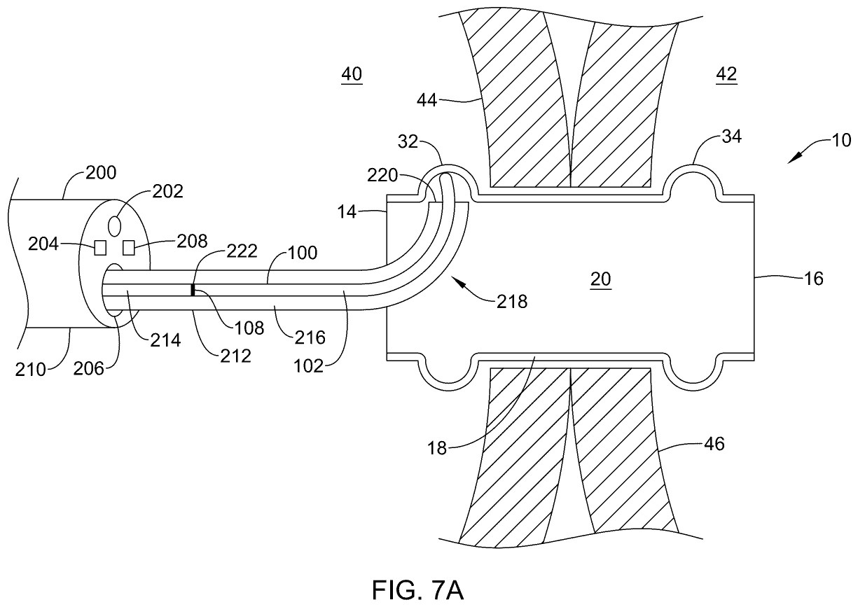

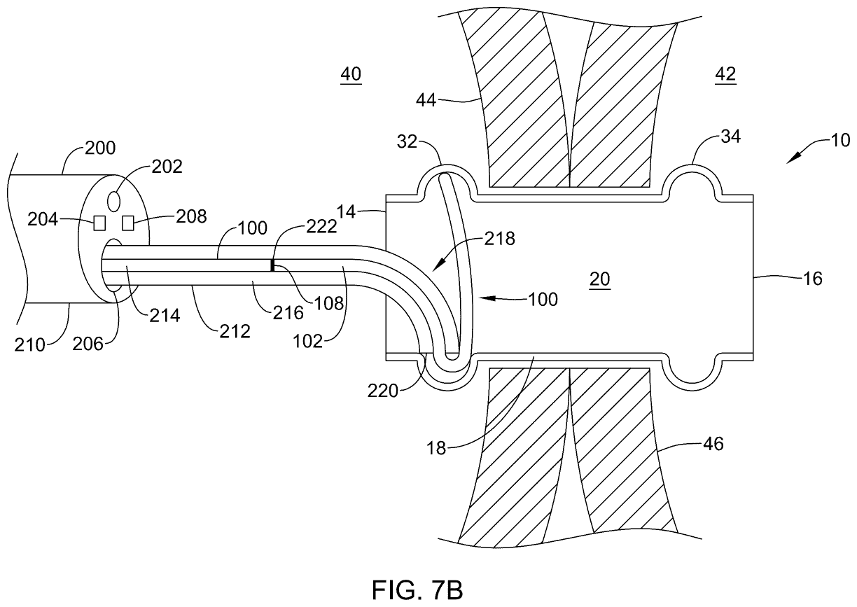

FIGS. 7A and 7B illustrate schematic partial cross-sectional views of an illustrative system and method for delivering the reinforcement member 100. In FIGS. 7A and 7B, a stent 10 has been delivered to the stomach 40 and an anastomosis has been formed between the stomach 40 and the jejunum 42. A proximal flange 32 is disposed adjacent to the stomach wall 44 and a distal flange 34 is disposed adjacent to the jejunum wall 46. As the flow of food particles, fluid, etc., moves from the stomach 40 to the jejunum 42, the stent 10 may be prone to migrate distally from the stomach 40 to the jejunum 42. Thus, a reinforcement member 100 may be positioned within the proximal flange 32 to make distal movement of the stent 10 more difficult. It is contemplated that the reinforcement member 100 may be delivered through an endoscope 200, which may be the same endoscope used to the deliver the stent 10. While the method and system to deliver the reinforcement member 100 is described with respect to a stent 10, it should be understood that the reinforcement member 100 may be delivered to the pyloric closure device 50 in a similar manner.

The endoscope 200 may include an elongated shaft 210 including a plurality of lumens. For example, the elongated shaft 210 may include an irrigation lumen 202 for receiving a flow of fluid, a lens wash lumen 204 for receiving a flow of fluid, a working channel 206 for receiving one or more devices therethrough, and an illumination channel 208. The elongated shaft 210 may include fewer than four or more than four channels or lumens, as desired. After the stent 10 has been deployed, a delivery shaft 212 may be advanced into or movable within the working channel 206. The delivery shaft 212 may extend from a distal end region 218 to a proximal end region (not explicitly shown) configured to remain outside the body. In some cases, the reinforcement member 100 may be preloaded within a lumen 216 of the delivery shaft 212. In other examples, the reinforcement member 100 may be positioned within the lumen 216 of the delivery shaft 212 at a time of implantation or a time of use. The delivery shaft 212 may have a stiffness that is greater than a stiffness of the reinforcement member 100 such that the delivery shaft 212 may maintain the reinforcement member 100 in a straightened, elongated configuration for delivery. In some embodiments, the distal end region 218 of the delivery shaft 212 may include a predefined curve. The curved distal end region 218 may be configured to direct the reinforcement member 100 into the flange 32 of the stent 10.

The delivery shaft 212 may be distally advanced through the working channel 206, until a distal end 220 of the delivery shaft 212 is adjacent to the proximal flange 32 (or other deployment location). Once the distal end 220 is adjacent to the target deployment location, a pusher element 214 may be distally advanced. The pusher element 214 may extend from a distal end 222 to a proximal end (not explicitly shown) configured to remain outside the body. The distal end 222 of the pusher element 214 may engage a proximal end 108 of the reinforcement member 100. As the pusher element 214 is distally advanced, the pusher element 214 pushes the reinforcement member 100 distally out of the distal end 220 of the delivery shaft 212. As the reinforcement member 100 exits the lumen 216 of the delivery shaft 212, the restraining force of the delivery shaft 212 is removed and the reinforcement member 100 resumes its curved or looped configuration. Said differently, the reinforcement member 100 may be configured to move from an elongated delivery configuration within the lumen 216 of the delivery shaft to a curved deployed configuration as the reinforcement member 100 is distally advanced from the delivery shaft 212. As the reinforcement member 100 is deployed, the delivery shaft 212 may rotate about a circumference of the flange 32, as shown in FIG. 7B.

In some embodiments, the pusher element 214 may be releasably coupled to the proximal end 108 of the wire 102. This may allow the reinforcement member 100 to be proximally retracted back into the lumen 216 of the delivery shaft 212 if repositioning and/or re-constrainment of the reinforcement member 100 is desired.

As noted above, the reinforcement member 100 may include a variety of different configurations. In some examples, a reinforcement member 100 having more than one winding 104 may be deployed using a single wire 102. In such an instance, the delivery shaft 212 may make more than one rotation about the flange 32 as the reinforcement member 100 is deployed. In other examples, a plurality of the wires 102 may be used to form the desired reinforcement member 100. In some cases, the plurality of wires 102 may be loaded simultaneously within the lumen 216 of the delivery shaft 212 and delivered one after the other without needed to remove the pusher element 214 and insert additional wires 102. In other examples, one or more wires 102 may be delivered and then one or more additional wires 102 may be supplied to the delivery shaft 212. It is contemplated that the one or more additional wires 102 may be loaded into a same delivery shaft 212 by removing the pusher element 214 and inserting the one or more wires 102. In another example, the one or more additional wires 102 may be provided in an additional delivery shaft 212 which is inserted into the working channel 206 after removal of the initial delivery shaft 212. When more than one wire 102 is used to form the reinforcement member 100, it is contemplated that the ends of the successive wires 102 may not overlap, may partially overlap (e.g., less than 50% of a length of the wire 102 overlaps a preceding or subsequent wire 102), or may substantially overlap (e.g., more than 50% of a length of the wire 102 overlaps a preceding or subsequent wire 102), as desired. In some cases, a plurality of wires 102 may be used to form one complete winding 104. In other cases, a plurality of wires 102 may be used to form more than one complete winding.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A transluminal implant, comprising:
an elongated tubular body extending from first end region to a second end region and comprising:
a scaffolding forming a plurality of cells;
a first flange adjacent to the first end region;
a second flange adjacent to the second end region; and
a saddle region extending between the first flange and the second flange, the saddle region having an outer diameter less than an outer diameter of the first flange and the second flange; and
a reinforcement member disposed within a lumen of the elongated tubular body and within the first flange, the reinforcement member comprising at least one curved wire.

2. The transluminal implant of claim 1, wherein the reinforcement member is configured to exert a radially outward force on the elongated tubular body.

3. The transluminal implant of claim 1, wherein an outer diameter of the reinforcement member is similar to an outer diameter of the first flange.

4. The transluminal implant of claim 1, wherein an outer diameter of the reinforcement member is greater than an outer diameter of the first flange.

5. The transluminal implant of claim 1, wherein the at least one curved wire includes at least one winding extending about an inner surface of the first flange.

6. The transluminal implant of claim 1, wherein the at least one curved wire includes at least two windings extending about an inner surface of the first flange.

7. The transluminal implant of claim 1, wherein the at least one curved wire includes less than one winding.

8. The transluminal implant of claim 1, wherein the reinforcement member comprises two or more curved wires.

9. The transluminal implant of claim 1, wherein the at least one curved wire has a diameter of about 0.013 inches (0.330 millimeters).

10. The transluminal implant of claim 1, wherein a proximal end of the at least one curved wire is secured to a distal end of the at least one curved wire.

11. The transluminal implant of claim 1, wherein the at least one curved wire comprises a shape memory material.

12. The transluminal implant of claim 1, further comprising a second reinforcement member disposed within the lumen of the elongated tubular body and within the second flange.

13. The transluminal implant of claim 1, wherein the elongated tubular body comprises a stent.

14. The transluminal implant of claim 1, wherein the elongated tubular body comprises a pyloric closure device.

15. The transluminal implant of claim 1, wherein the reinforcement member further comprises a hardening material.

16. A transluminal implant, comprising:
an elongated tubular body extending from first end region to a second end region and comprising:
a scaffolding forming a plurality of cells;
a first flange adjacent to the first end region;
a second flange adjacent to the second end region; and
a saddle region extending between the first flange and the second flange, the saddle region having an outer diameter less than an outer diameter of the first flange and the second flange; and
a self-expanding reinforcement member disposed against an inner surface of the elongated tubular body within the first flange, the self-expanding reinforcement member configured to exert a radially outward force on the elongated tubular body.

17. A delivery system for delivering a reinforcement member to an implant, the delivery system comprising:
an endoscope including a working channel;
a delivery shaft movably disposed within the working channel;
a reinforcement member disposed within a lumen of the delivery shaft; and
a pusher element movably disposed within the lumen of the delivery shaft, a distal end of the pusher element configured to engage a proximal end of the reinforcement member.

18. The delivery system of claim 17, wherein the delivery shaft has a stiffness greater than a stiffness of the reinforcement member.

19. The delivery system of claim 17, wherein the reinforcement member is configured to move from an elongated delivery configuration to a curved deployed configuration as the reinforcement member is distally advanced from the delivery shaft.

20. The delivery system of claim 17, wherein the delivery shaft includes a curved distal end region.
